(19) United States
(12) Patent Application Publication
(10) Pub. No.: US 2014/0271745 A1
(43) Pub. Date: Sep. 18, 2014

(54) Personal Care Article Comprising Dissolvable Fibers

(71) Applicant: The Procter & Gamble Company, Cincinnati, OH (US)

(72) Inventors: Robert Wayne GLENN, JR., Liberty Twp, OH (US); Todd Ryan THOMPSON, Loveland, OH (US); Ungyeong JUNG, North Royalton, OH (US); Chul B. PARK, Etobicoke (CA); Changwei ZHU, Richmond Hill (CA); Raymond Kar Man CHU, Markham (CA)

(73) Assignee: The Procter & Gamble Company, Cincinnati, OH (US)

(21) Appl. No.: 14/211,943

(22) Filed: Mar. 14, 2014

Related U.S. Application Data
(60) Provisional application No. 61/794,819, filed on Mar. 15, 2013.

(51) Int. Cl. A61K 8/02; A61Q 19/00; A61Q 5/12; A61Q 5/00; A61Q 5/02; A61Q 19/10

(52) U.S. Cl. CPC A61K 8/0216; A61Q 5/02; A61Q 19/10; A61Q 5/12; A61Q 5/006; A61Q 19/007
USPC 424/401; 510/451; 424/70.12; 424/70.122; 514/772.3

(57) ABSTRACT

Provided is a personal care article including one or more extruded dissolvable fibers. The extruded dissolvable fibers include (a) from about 10% to about 60% of one or more anionic surfactants; (b) from about 10% to about 50% of one or more water soluble polymers; (c) from about 1% to about 30% of one or more plasticizers; and (d) from about 0.01% to about 30% water. The one or more anionic surfactants have a Krafft point of less than about 30° C. The one or more extruded dissolvable fibers has an average diameter of from about 20 microns to about 1,000 microns. The personal care article has a dry density of from about 0.02 g/cm³ to about 0.30 g/cm³.

### FIELD OF THE INVENTION

The present invention relates to a personal care article comprising one or more extruded dissolvable fibers. The extruded dissolvable fibers comprise an anionic surfactant, a water soluble polymer, a plasticizer, and water.

### BACKGROUND OF THE INVENTION

Solid soaps are generally harsh and lead to a squeaky feel on the skin and hair. These qualities are generally unacceptable for many of today's consumers.

Anionic surfactants such as alkyl ether sulfates have been developed to improve upon the disadvantages of solid soaps. However, many anionic surfactants have low Krafft points and are thereby generally formulated only in liquid products. This is one of the primary reasons for the proliferation of liquid shampoos and liquid body washes across the personal care industry. While widely used, liquid products have disadvantages in terms of packaging, storage, transportation, and convenience of use.

To address the disadvantages of liquid products, attempts have been made to incorporate the benefits of low Krafft point anionic surfactants into dissolvable solids. One attempt was to structure the dissolvable solid with one or more water soluble polymers via a casting and drying process. However, this process was energy intensive and costly because it involves the drying of significant amounts of water (typically >50%).

Another attempt was to create porous solids comprising low Krafft point anionic surfactants by freeze-drying. However, freeze-drying was also an energy intensive and costly process.

Producing a dissolvable personal care article via extrusion is a challenge due to the hydrolytic degradation of low Krafft point anionic surfactants under high temperature extrusion conditions. Additionally, low Krafft point anionic surfactants are typically available as aqueous “lamellar” pastes (comprising ˜30% water) and impart significant lubricity inside the extruder barrel which significantly limits the friction and torque between the mixing elements and the extruder barrel, inhibiting the ability of the extruder to work effectively. Moreover, the large viscosity difference between low Krafft point anionic surfactants (as available commercially) and water soluble polymers imposes significant mixing challenges.

Some dissolvable fibers comprising water soluble polymers and low Krafft point anionic surfactants are known. However, these fibers are spun and dried from aqueous solutions and are accordingly very fine with small diameters. Such fine fibers are generally too difficult to handle by the consumer on their own (sticky like cotton candy) and also too weak to assemble into low density 3-D porous web structures. Low density 3-D porous structures are desired which have superior dissolution properties and markedly less propensity for gel blocking. Gel blocking, occurs when there is insufficient permeation of water throughout the substrate during the dissolution process due to the formation of localized hydrated gels or highly viscous concentrated regions. These formations block the pores and thereby restrict subsequent water penetration, forming clumps or pieces that do not fully dissolve.

Based on the forgoing, there is a need for a personal care article comprising one or more extruded dissolvable fibers with a diameter large enough for assembling into the personal care article.

### SUMMARY OF THE INVENTION

According to an embodiment of the invention, there is provided a personal care article comprising one or more extruded dissolvable fibers, the extruded dissolvable fibers comprising (a) from about 10% to about 60% of one or more anionic surfactants, wherein the one or more anionic surfactants have a Krafft point of less than about 30° C.; (b) from about 10% to about 50% of one or more water soluble polymers; (c) from about 1% to about 30% of one or more plasticizers; and (d) from about 0.01% to about 30% water; wherein the one or more extruded dissolvable fibers has an average diameter of from about 20 microns to about 1,000 microns; and wherein the personal care article has a dry density of from about 0.02 g/cm³ to about 0.30 g/cm³.

These and other features, aspects, and advantages of the invention will become evident to those skilled in the art from a reading of the following disclosure.

### DETAILED DESCRIPTION OF THE INVENTION

While the specification concludes with claims which particularly point out and distinctly claim the invention, it is believed the present invention will be better understood from the following description.

In all embodiments of the present invention, all percentages are by weight of the total composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

The term “comprising,” as used herein, means that other steps and other ingredients which do not affect the end result can be added. This term encompasses the terms “consisting of” and “consisting essentially of.” The compositions and methods/processes of the present invention can comprise, consist of, and consist essentially of the elements and limitations of the invention described herein, as well as any of the additional or optional ingredients, components, steps, or limitations described herein.

The term “extruded,” as used herein, means having been produced from the basic components of an extrusion line including a polymer feed, the extruder drive and gear box, the extruder barrel with one or two screws, one or more other injection ports, and the extrusion die. The extruder drive may be electrical in operation and may be geared via a thrust bearing to produce the rotational movement of the one or two extruder screws. The polymer feed to the screw may be from the feed hopper and the feed may be by gravity, metering screw, or simple conveying spiral. The extruder barrel and one or two extruder screws are of high strength steels and are protected from wear and corrosion by a variety of hardening and coating treatments such as nitriding and hard chroming. The extrusion barrel and screw are zoned into between 3 and 15 sections which are individually heated and cooled depending on the material and process parameters. The extrusion die channels the polymer melt from the front of the one or two extruder screws to form the basic shape of the desired product.

The term “Krafft point,” as used herein, (also known as Krafft temperature, or critical micelle temperature) means the minimum temperature at which surfactants form micelles. Below the Krafft point, there is no value for the critical micelle concentration (CMC), i.e., micelles cannot form. The Krafft point is a point of phase change below which the surfactant remains in crystalline form, even in aqueous solution. The Krafft point is measured experimentally as the temperature (more precisely, narrow temperature range) above which the solubility of a surfactant rises sharply. At this temperature, the solubility of the surfactant becomes equal to the critical micelle concentration. The Krafft point of a surfactant is best determined by locating the abrupt change in slope of a graph of the logarithm of the surfactant's solubility versus temperature [Source: PAC, 1972, 31, 577 (Manual of Symbols and Terminology for Physicochemical Quantities and Units, Appendix II: Definitions, Terminology and Symbols in Colloid and Surface Chemistry) on page 613].

The term “plasticizer,” as used herein, means any of various substances (typically a solvent) added to a polymer composition to reduce brittleness and to promote plasticity and flexibility.

The term “semi-solid,” as used herein, means a state of matter which is highly viscous and has the qualities of both a solid and a liquid.

The term “solid,” as used herein, means a state of matter wherein the constituents are arranged such that their shape and volume are relatively stable, i.e., not liquid-like or gaseous.

The term “water soluble polymer,” as used herein, includes both water-soluble and water-dispersible polymers, and is defined as a polymer with a solubility in water, measured at 25° C., of at least about 0.1 gram/liter (g/L).

Provided is a personal care article comprising one or more extruded dissolvable fibers, the extruded dissolvable fibers comprising (a) from about 10% to about 60% of one or more anionic surfactants, wherein the one or more anionic surfactants have a Krafft point of less than about 30° C.; (b) from about 10% to about 50% of one or more water soluble polymers; (c) from about 1% to about 30% of one or more plasticizers; and (d) from about 0.01% to about 30% water; wherein the one or more extruded dissolvable fibers has an average diameter of from about 20 microns to about 1,000 microns; and wherein the personal care article has a dry density of from about 0.02 g/cm³ to about 0.30 g/cm³. The fibers may be formed from, not coated with or impregnated with after formation, the one or more anionic surfactants, the one or more water soluble polymers, the one or more plasticizers, and water.

The one or more extruded dissolvable fibers may have an average diameter of from about 20 microns to about 1,000 microns, alternatively from about 30 microns to about 500 microns, alternatively from about 40 microns to about 250 microns, alternatively from about 50 microns to about 150 microns, and alternatively from about 60 microns to about 130 microns.

The diameter of the one or more dissolvable fibers may be determined by placing a dissolvable fiber under an optical microscope. The diameter of the dissolvable fiber may be measured using a calibrated reticle and an objective of 100 power. The diameter may be read in at least 3 positions (in the center of the visible fiber and at 2 or more positions along the length of the fiber near opposite boundaries of the viewing area). The diameter may be taken as the largest dimension perpendicular to the optical microscope viewing axis. The diameter measurements at the 3 or more positions is averaged and reported as the average diameter of the dissolvable fiber.

In an embodiment, the one or more dissolvable fibers may be a shaped fiber. More specifically, the one or more dissolvable fibers may be multi-lobal. Non-limiting examples of shaped fibers may be selected from the group consisting of crescent shaped, oval shaped, square shaped, diamond shaped, and combinations thereof. Other suitable shapes may also be used. In an embodiment, the dissolvable fibers may be multi-lobal fibers having more than one critical point along the outer surface of the fiber. A critical point is defined as being a change in the absolute value of the slope of a line drawn perpendicular to the surface of the fiber when the fiber is cut perpendicular to the fiber axis. Solid round fibers have an optically continuous distribution of matter across the width of the fiber cross section. These fibers may contain microvoids or internal fibrillation but may be recognized as being substantially continuous. There may be no critical points for the exterior surface of solid round fibers.

In an embodiment, the fibers are trilobal in shape with a modification ratio of at least 1.4. The modification ratio may also be from about 1.4 to about 8, alternatively from about 1.5 to about 7, and alternatively from about 2.0 to about 5. The modification ratio may be the ratio R1/R2 where R2 is the radius of the largest circle that is wholly within a transverse cross section of the fiber, and R1 is the radius of the circle that circumscribes the transverse cross-section.

The personal care article may have a dry density of from about 0.02 g/cm³ to about 0.30 g/cm³, alternatively from about 0.06 g/cm³ to about 0.20 g/cm³, and alternatively from about 0.08 g/cm³ to about 0.15 g/cm³.

### Anionic Surfactant

The personal care article may comprise from about 10% to about 60%, alternatively from about 12% to about 50%, and alternatively from about 15% to about 40% of one or more anionic surfactants, by weight of the personal care article. The one or more anionic surfactants may have a Krafft point of less than 30° C., alternatively less than 25° C., alternatively less than 20° C., alternatively less than 15° C., and alternatively less than 10° C.

Non-limiting examples of anionic surfactants may be selected from the group consisting of alkyl sulfates, alkyl ether sulfates, branched alkyl sulfates, branched alkyl alkoxylates, branched alkyl alkoxylate sulfates, alkyloxy alkane sulfonates mid-chain branched alkyl aryl sulfonates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, sodium lauroyl glutamate, and combinations thereof.

In an embodiment, the one or more anionic surfactants may comprise one or more alkyl ether sulfates according to the following structure:

[chemical structure]

wherein R¹ is a C-linked monovalent substituent selected from the group consisting of:
a. substituted alkyl systems comprising from about 9 to about 15 carbon atoms;
b. unsubstituted alkyl systems comprising from about 9 to about 15 carbon atoms;
c. straight alkyl systems comprising from about 9 to about 15 carbon atoms;
d. branched alkyl systems comprising from about 9 to about 15 carbon atoms; and
e. unsaturated alkyl systems comprising from about 9 to about 15 carbon atoms;
   wherein R² is selected from the group consisting of:
a. C-linked divalent straight alkyl systems comprising from about 2 to about 3 carbon atoms;
b. C-linked divalent branched alkyl systems comprising from about 2 to about 3 carbon atoms; and
c. combinations thereof;
   wherein M+ is a monovalent counterion selected from a group consisting of sodium, potassium, ammonium, protonated monoethanolamine, protonated diethanolamine, and protonated triethanolamine; and wherein x is on average of from about 0.5 moles to about 3 moles, alternatively from about 1 mole to about 2 moles. In an embodiment, x is on average from about 0.5 moles to about 3 moles of ethylene oxide, alternatively from about 1 mole to about 2 moles of ethylene oxide.

Alkyl sulfates suitable for use herein include materials with the respective formula ROSO₃M, wherein R is an alkyl or an alkenyl of from about 8 carbon atoms to about 24 carbon atoms, and M is a water-soluble cation. Non-limiting examples of M may be selected from the group consisting of ammonium, sodium, potassium, and triethanolamine.

Non-limiting examples of alkyl ether sulfates may be selected from the group consisting of sodium laureth sulfates, ammonium laureth sulfates, potassium laureth sulfates, triethanolamine laureth sulfates, sodium trideceth sulfates, ammonium trideceth sulfates, potassium trideceth sulfates, triethanolamine trideceth sulfates, sodium undeceth sulfates, ammonium undeceth sulfates, potassium undeceth sulfates, triethanolamine undeceth sulfates, and combinations thereof. In an embodiment, the alkyl ether sulfate may be sodium laureth sulfates.

Other suitable anionic surfactants may be described in McCutcheon's Detergents and Emulsifiers, North American Edition (1986), Allured Publishing Corp.; McCutcheon's Functional Materials, North American Edition (1992), Allured Publishing Corp; and U.S. Pat. Nos. 2,486,921, 2,486,922, and 2,396,278.

### Secondary Surfactant

The personal care article may further comprise one or more secondary surfactants selected from the group consisting of amphoteric surfactants, zwitterionic surfactants, and mixtures thereof. The ratio of the one or more anionic surfactants to the one or more secondary surfactants may be from about 15:1 to about 1:2, alternatively from about 10:1 to about 1:1.

Non-limiting examples of amphoteric surfactants may be selected from the group consisting of aliphatic derivatives of secondary and tertiary amines, aliphatic derivatives of heterocyclic secondary and tertiary amines, and mixtures thereof.

Further non-limiting examples of amphoteric surfactants may be selected from the group consisting of sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethy-7 carboxyamphodiacetate, lauraminopropionic acid, lauroamphodipropionic acid, lauryl aminopropylglycine, lauryl diethylenediaminoglycine, and mixtures thereof.

Non-limiting examples of zwitterionic surfactants may be selected from the group consisting of derivatives of secondary and tertiary amines, derivatives of heterocyclic secondary and tertiary amines, derivatives of quaternary ammonium, derivatives of quaternary phosphonium, derivatives of tertiary sulfonium, and mixtures thereof.

Non-limiting examples of zwitterionic surfactants may also be selected from the group consisting of betains including alkyl dimethyl betaine and cocodimethyl amidopropyl betaine, C₈-C₁₈ amine oxides, sulfo and hydroxy betaines, and mixtures thereof.

Further non-limiting examples of zwitterionic surfactants may be selected from the group consisting of cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, oleamidopropyl betaine, coco-sultaine, lauramidopropyl betaine, lauryl betaine, lauryl hydroxysultaine, lauryl sultaine, and mixtures thereof.

### Water-Soluble Polymer

The personal care article may comprise one or more water soluble polymers that may function as a structurant. The personal care article may comprise from about 10% to about 50%, alternatively from about 15% to about 45%, alternatively from about 20% to about 40%, and alternatively from about 25% to about 35% of one or more water soluble polymers, by weight of the personal care article.

The one or more water soluble polymers may have solubility in water, measured at 25° C., of from about 0.1 g/L to about 500 g/L. The one or more water soluble polymers may be of synthetic or natural origin and may be modified by means of a chemical reaction.

In an embodiment, the one or more water soluble polymers may have a weight average molecular weight of from about 40,000 g/mol to about 500,000 g/mol, alternatively from about 50,000 g/mol to about 400,000 g/mol, alternatively from about 60,000 g/mol to about 300,000 g/mol, and alternatively from about 70,000 g/mol to about 200,000 g/mol.

In an embodiment, a 4% by weight solution of one or more water soluble polymers may have a viscosity at 20° C. of from about 4 centipoise to about 80 centipoise, alternatively from about 10 centipoise to about 60 centipoise, and alternatively from about 20 centipoise to about 40 centipoise.

Non-limiting examples of synthetic water soluble polymers may be selected from the group consisting of polyvinyl alcohols, polyvinylpyrrolidones, polyalkylene oxides, polyacrylates, caprolactams, polymethacrylates, polymethylmethacrylates, polyacrylamides, polymethylacrylamides, polydimethylacrylamides, polyethylene glycol monomethacrylates, polyurethanes, polycarboxylic acids, polyvinyl acetates, polyesters, polyamides, polyamines, polyethyleneimines. Further non-limiting examples of synthetic water soluble polymers may be selected from the group consisting of copolymers of anionic, cationic and amphoteric monomers and mixtures thereof, including maleic acrylate based copolymers, maleic methacrylate based copolymers, copolymers of methylvinyl ether and of maleic anhydride, copolymers of vinyl acetate and of crotonic acid, copolymers of vinylpyrrolidone and of vinyl acetate, and copolymers of vinylpyrrolidone and of caprolactam.

Non-limiting examples of natural water soluble polymers may be selected from the group consisting of karaya gum, tragacanth gum, gum arabic, acemannan, konjac mannan, acacia gum, gum ghatti, whey protein isolate, soy protein isolate, guar gum, locust bean gum, quince seed gum, psyllium seed gum, carrageenan, alginates, agar, fruit extracts (pectins), xanthan gum, gellan gum, pullulan, hyaluronic acid, chondroitin sulfate, and dextran, casein, gelatin, keratin, keratin hydrolysates, sulfonic keratins, albumin, collagen, glutelin, glucagons, gluten, zein, shellac, and mixtures thereof.

Non-limiting examples of modified natural water soluble polymers may be selected from the group consisting of (1) cellulose derivatives including hydroxypropylmethylcellulose, hydroxymethylcellulose, hydroxyethylcellulose, methylcellulose, hydroxypropylcellulose, ethylcellulose, carboxymethylcellulose, cellulose acetate phthalate, nitrocellulose, cellulose ethers, cellulose esters; and (2) guar derivatives including hydroxypropyl guar. Suitable hydroxypropylmethylcelluloses may include those available from the Dow Chemical Company (Midland, Mich.).

In an embodiment, the one or more water soluble polymers may be blended with a starch-based material in such an amount as to reduce the overall level of water soluble polymer required. The combined weight percentage of the one or more water soluble polymers and the starch-based material may range from about 10% to about 40%, alternatively from about 12% to about 30%, and alternatively from about 15% to about 25%, by weight of the personal care article. The weight ratio of the one or more water soluble polymers to the starch-based material may range from about 1:10 to about 10:1, alternatively from about 1:8 to about 8:1, alternatively from about 1:7 to about 7:1, and alternatively from about 6:1 to about 1:6.

Non-limiting examples of starch-based materials may be selected from the group consisting of cereals, tubers, roots, legumes, fruits, and combinations thereof. More specifically, non-limiting examples of starch-based materials may be selected from the group consisting of corn, peas, potatoes, bananas, barley, wheat, rice, sago, amaranth, tapioca, arrowroot, canna, sorghum, and combinations thereof. The starch-based materials may also include native starches that are modified using any modification known in the art, including physically modified starches and chemically modified starches.

### Plasticizer

The personal care article may comprise one or more plasticizers. The personal care article may comprise from about 1% to about 30%, alternatively from about 5% to about 25%, and alternatively from about 10% to about 20% of one or more plasticizers, by weight of the personal care article. Non-limiting examples of plasticizers may be selected from the group consisting of polyols, copolyols, polycarboxylic acids, polyesters, dimethicone copolyols, and mixtures thereof.

Non-limiting examples of suitable polyols may be selected from the group consisting of glycerin, diglycerin, propylene glycol, ethylene glycol, butylene glycol, pentylene glycol, cyclohexane dimethanol, hexanediol, polyethylene glycol, sorbitol, manitol, lactitol, monohydric and polyhydric low molecular weight alcohols (e.g., C₂-C₈ alcohols), monosaccharides, disaccharides, oligosaccharides, high fructose corn syrup solids, ascorbic acid, and mixtures thereof.

Non-limiting examples of suitable polycarboxylic acids may be selected from the group consisting of citric acid, maleic acid, succinic acid, polyacrylic acid, polymaleic acid, and mixtures thereof.

Non-limiting examples of suitable polyesters may be selected from the group consisting of glycerol triacetate, acetylated-monoglyceride, diethyl phthalate, triethyl citrate, tributyl citrate, acetyl triethyl citrate, acetyl tributyl citrate, and mixtures thereof.

Non-limiting examples of suitable dimethicone copolyols may be selected from the group consisting of PEG-12 dimethicone, PEG/PPG-18/18 dimethicone, and PPG-12 dimethicone.

Further non-limiting examples of suitable plasticizers may be selected from the group consisting of alkyl phthalates, allyl phthalates, napthalates, lactates (e.g., sodium, ammonium and potassium salts), sorbeth-30, urea, lactic acid, sodium pyrrolidone carboxylic acid (PCA), sodium hyaluronate, hyaluronic acid, soluble collagen, modified protein, monosodium L-glutamate, glyceryl polymethacrylate, polymeric plasticizers, proteins, amino acids, hydrogen starch hydrolysates, low molecular weight esters (e.g., esters of C₂-C₁₀ alcohols and acids), and mixtures thereof. In an additional embodiment, non-limiting examples of suitable plasticizers may be alpha and beta hydroxyl acids selected from the group consisting of glycolic acid, lactic acid, citric acid, maleic acid, salicylic acid, and mixtures thereof. EP 0283165 B1 discloses even more suitable plasticizers, including glycerol derivatives such as propoxylated glycerol.

### Water

The personal care article may comprise from about 0.01% to about 30%, alternatively from about 1% to about 20%, alternatively from about 2% to about 15% water, by weight of the personal care article.

### Benefit Agent

The personal care article may comprise from about 0.1% to about 15% of a benefit agent. Non-limiting examples of suitable benefit agents may be selected from the group consisting of nonionic surfactants, preservatives, perfumes, coloring agents, cationic polymers, conditioning agents, hair bleaching agents, thickeners, moisturizers, emollients, pharmaceutical actives, vitamins, sunscreens, deodorants, sensates, plant extracts, cosmetic particles, reactive agents, skin lightening agents, skin tanning agents, anti-dandruff agents, exfoliating agents, acids, bases, humectants, enzymes, suspending agents, pH modifiers, hair perming agents, anti-acne agents, anti-microbial agents, exfoliation particles, hair growth agents, insect repellents, chelants, dissolution aids, builders, enzymes, dye transfer inhibiting agents, softening agents, and mixtures thereof.

In an embodiment, the personal care article may be configured as a lubricating strip on a disposable shaving device.

### Conditioning Agent

Non-limiting examples of conditioning agents may be selected from the group consisting of silicones, organic oils, and mixtures thereof. Non-limiting examples of silicones may be selected from the group consisting of silicone oils, high molecular weight polyalkyl or polyaryl siloxanes, aminosilicones, cationic silicones, silicone gums, high refractive silicones, low molecular weight polydimethyl siloxanes, silicone resins, and mixtures thereof. Non-limiting examples of organic oils may be selected from the group consisting of hydrocarbon oils, polyolefins, fatty esters, and mixtures thereof. Additional non-limiting examples of conditioning agents and optional suspending agents for silicone may be found in U.S. Pat. Nos. 5,104,646 and 5,106,609, which are incorporated herein by reference.

The silicone gums and the high molecular weight polyalkyl or polyaryl siloxanes may have a viscosity of from about 100,000 mPa·s to about 30,000,000 mPa·s, alternatively from about 200,000 mPa·s to about 30,000,000 mPa·s. The silicone gums and the high molecular weight polyalkyl or polyaryl siloxanes may have a molecular weight of from about 100,000 g/mol to about 1,000,000 g/mol, and alternatively from about 120,000 g/mol to about 1,000,000 g/mol.

The low molecular weight polydimethyl siloxanes may have a viscosity of from about 1 mPa·s to about 10,000 mPa·s at 25° C., and alternatively from about 5 mPa·s to about 5,000 mPa·s. The low molecular weight polydimethyl siloxanes may have a molecular weight of from about 400 to about 65,000, and alternatively from about 800 to about 50,000.

In an embodiment, the conditioning agent may include one or more aminosilicones. Aminosilicones may be silicones containing at least one primary amine, secondary amine, tertiary amine, or a quaternary ammonium group. In an embodiment the aminosilicones may have less than about 0.5% nitrogen by weight of the aminosilicone, in another embodiment less than about 0.2%, in yet another embodiment less than about 0.1%.

The aminosilicones may have a viscosity of from about 1,000 cs (centistokes) to about 1,000,000 cs, in another embodiment from about 10,000 cs to about 700,000 cs, in yet another embodiment from about 50,000 cs to about 500,000 cs, and in yet another embodiment from about 100,000 cs to about 400,000 cs. This embodiment may also comprise a low viscosity fluid. The viscosity of aminosilicones discussed herein is measured at 25° C.

In another embodiment, the aminosilicones may have a viscosity of from about 1,000 cs to about 100,000 cs, in another embodiment from about 2,000 cs to about 50,000 cs, in another embodiment from about 4,000 cs to about 40,000 cs, and in yet another embodiment from about 6,000 cs to about 30,000 cs.

The personal care composition may comprise from about 0.05% to about 20%, alternatively from about 0.1% to about 10%, and alternatively from about 0.3% to about 5% aminosilicones by weight of the personal care composition.

### Anti-Dandruff Agent

In an embodiment, the personal care article may comprise an anti-dandruff agent which may be an anti-dandruff particulate. Non-limiting examples of suitable anti-dandruff agents may be selected from the group consisting of pyridinethione salts, azoles (e.g. ketoconazole, econazole, and elubiol), selenium sulphide, particulate sulfur, keratolytic agents (e.g. salicylic acid), and mixtures thereof. In an embodiment, the anti-dandruff agent is a pyridinethione salt.

Pyridinethione salt particulates are suitable particulate anti-dandruff agents. In an embodiment, the anti-dandruff agent may be a 1-hydroxy-2-pyridinethione salt in particulate form. The personal care article may comprise from about 0.01% to about 5%, alternatively from about 0.1% to about 3%, and alternatively from about 0.1% to about 2% pyridinethione salt particulates. In an embodiment, the pyridinethione salt particulates may be those formed from heavy metals such as zinc, tin, cadmium, magnesium, aluminium, and zirconium. In any embodiment, the pyridinethione salt may be the zinc salt of 1-hydroxy-2-pyridinethione (known as “zinc pyridinethione” or “ZPT”) optionally in platelet particle form. In an embodiment, the zinc salt of 1-hydroxy-2-pyridinethione in platelet particle form may have an average particle size of less than 20 microns, alternatively less than 5 microns, and alternatively less than 2.5 microns. Salts formed from other cations, such as sodium, may also be suitable anti-dandruff agents. Pyridinethione anti-dandruff agents are described, for example, in U.S. Pat. Nos. 4,323,683; 4,379,753; and 4,470,982.

The personal care article may also comprise an antimicrobial active. Non-limiting examples of suitable anti-microbial actives may be selected from the group consisting of coal tar, sulfur, charcoal, aluminum chloride, gentian violet, octopirox (piroctone olamine), ciclopirox olamine, undecylenic acid and its metal salts, potassium permanganate, selenium sulphide, sodium thiosulfate, propylene glycol, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, azoles, and mixtures thereof. Further non-limiting examples of suitable anti-microbial agents may be selected from the group consisting of itraconazole, ketoconazole, selenium sulphide, coal tar, and mixtures thereof.

In an embodiment, the anti-microbial agent may be an imidazole selected from the group consisting of benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof. In an embodiment, the anti-microbial agent may be a triazole selected from the group consisting of terconazole, itraconazole, and mixtures thereof.

### Cationic Polymer

In an embodiment, the personal care article may comprise a cationic polymer. Cationic polymers useful herein may include those discussed in US 2007/0207109 A1 and US 2008/0206185 A1, such as synthetic copolymers of sufficiently high molecular weight to effectively enhance the deposition of the conditioning active components of the personal care article described herein. Combinations of cationic polymer may also be utilized. The average molecular weight of the synthetic copolymers is generally between about 10,000 and about 10 million, preferably between about 100,000 and about 3 million, still more preferably between about 200,000 and about 2 million.

In a further embodiment, the synthetic copolymers have mass charge densities of from about 0.1 meq/gm to about 6.0 meq/gm, alternatively from about 0.5 meq/gm to about 3.0 meq/gm, at the pH of intended use of the personal care article. The pH may be from about pH 3 to about pH 9, and alternatively from about pH 4 and about pH 8.

In yet another embodiment, the synthetic copolymers have linear charge densities from at least about 2 meq/A to about 500 meq/A, and more preferably from about 20 meq/A to about 200 meq/A, and most preferably from about 25 meq/A to about 100 meq/A.

Cationic polymer may be copolymers or homopolymers. In one embodiment, a homopolymer is utilized in the present composition. In another embodiment, a copolymer is utilized in the present composition. In another embodiment a mixture of a homopolymer and a copolymer is utilized in the present composition. In another embodiment, a homopolymer of a naturally derived nature, such as cellulose or guar polymer discussed herein, is combined with a homopolymer or copolymer of synthetic origin, such as those discussed below.

Homopolymers—Non-crosslinked cationic homopolymers of the following monomers are also useful herein: 3-acrylamidopropyltrimethylammonium chloride (APTAC), diallyldimethylammonium chloride (DADMAC), [(3-methylacrylolyamino)propyl]trimethylammonium chloride (MAPTAC), 3-methyl-1-vinylimidazolium chloride (QVI); [2-(acryloyloxy)ethyl]trimethylammonium chloride and [2-(acryloyloxy)propyl]trimethylammonium chloride.

Copolymers—copolymer may be comprises of two cationic monomer or a nonionic and cationic monomers.

The personal care articles may also comprise cellulose or guar cationic deposition polymers. Generally, such cellulose or guar cationic deposition polymers may be present at a concentration from about 0.05% to about 5%, by weight of the composition. Suitable cellulose or guar cationic deposition polymers have a molecular weight of greater than about 5,000. Additionally, such cellulose or guar deposition polymers have a charge density from about 0.5 meq/g to about 4.0 meq/g at the pH of intended use of the personal care article, which pH will generally range from about pH 3 to about pH 9, preferably between about pH 4 and about pH 8. The pH of the compositions is measured neat.

In one embodiment of the invention, the cationic polymers are derivatives of Hydroxypropyl Guar, examples of which include polymers known via the INCI nomenclature as Guar Hydroxypropyltrimonium Chloride, such as the products sold under the name Catinal CG-100, Catinal CG-200 by the company Toho, Cosmedia Guar C-261N, Cosmedia Guar C-261N, Cosmedia Guar C-261N by the company Cognis, DiaGum P 5070 by the company Freedom Chemical Diamalt, N-Hance Cationic Guar by the company Hercules/Aqualon, Hi-Care 1000, Jaguar C-17, Jaguar C-2000, Jaguar C-13S, Jaguar C-14S, Jaguar Excel by the company Rhodia, Kiprogum CW, Kiprogum NGK by the company Nippon Starch.

### Process of Forming the Personal Care Article

The one or more dissolvable fibers may be assembled into a personal care article having a dry density of from about 0.02 g/cm³ to about 0.30 g/cm³, alternatively from about 0.06 g/cm³ to about 0.20 g/cm³, and alternatively from about 0.08 g/cm³ to about 0.15 g/cm³. The personal care article may be assembled by any known processing means capable of bonding the dissolvable fibers or filaments together mechanically, thermally, or chemically to form a web structure.

In an embodiment, the one or more dissolvable fibers may be cut into lengths of from about 1 cm to about 40 cm, alternatively from about 2 cm to about 30 cm, and alternatively from about 3 cm to about 20 cm and put into bales. The one or more dissolvable fibers within the bales may be transported by mechanical and pneumatic processes into various web-forming machines. The feed system to the web-forming machine may be selected based on the type of dissolvable fiber and the type of web-former. Chute feeding may be used to feed fibers up to 6 cm in length. For longer fibers, a hopper feed with a shaker-type chute may be used.

In an embodiment, the web formation may be via a mechanical process such as carding or garnetting. In carding, the cut fibers may be held by one surface while the other surface combs the fibers causing individual fiber separation. At its center may be a large rotating metallic cylinder covered with card clothing. The card clothing may be comprised of needles, wires, or fine metallic teeth embedded in a heavy cloth or in a metallic foundation. The cylinder may be partly surrounded by an endless belt of a large number of narrow, cast iron flats positioned along the top of the cylinder. The top of the cylinder may be covered by alternating rollers and stripper rolls in a roller-top card. In garnetting, a group of rolls may be placed in an order that allows a given wire configuration, along with certain speed relationships, to level, transport, comb and interlock the cut fibers to a degree that a web is formed. Garnetting may deliver a more random web than a card. Webs from garnetts may be layered by crosslapping to build up the desired finished nonwoven weight.

In an embodiment, the web formation may be via an aerodynamic process such as the air-lay process. In an air-lay process, the cut fibers may be captured on a screen from an air stream. The length of fibers used in air-laying may vary from 2 cm to 6 cm. The web may then be delivered to a conveyor for transporting to the bonding area.

In an embodiment, the web formation may be via a centrifugal dynamic web formation process. Here the web may be formed via a centrifugal dynamic random card which forms a web by throwing off fibers from the cylinder onto a doffer with fiber inertia, which is subject to centrifugal force, in proportion to the square of the rotary speed.

In an embodiment, the above web formations may be made into the desired web structure by the layering of the webs. Layering can be accomplished in several ways to reach the desired weight and web structure. In an embodiment, longitudinal layering may be employed whereby carded webs from all the cards (placed in a sequence one after the other) are laid above one another on a conveyor belt and later bonded. In an embodiment, cross layering may be employed using two different devices (cross lappers)—a vertical and a horizontal cross lapper. In an embodiment, perpendicular layering may also be employed.

In an embodiment, the above webs may be bonded via various means including mechanical bonding (needle punching, stitch bonding), thermal bonding, chemical bonding, and hydroentanglement.

The personal care article may be dissolvable. As used herein, “dissolvable” means that the personal care article meets the hand dissolution values discussed herein. The personal care article may have a hand dissolution value of from about 1 to about 30 strokes, alternatively from about 2 to about 25 strokes, alternatively from about 3 to about 20 strokes, and alternatively from about 4 to about 15 strokes, as measured by the Hand Dissolution Method below.

### Hand Dissolution Method

One personal care article, with dimensions of approximately 43 mm×43 mm×4-6 mm, is placed in the palm of the hand while wearing nitrile gloves. 7.5 cm³ of from about 30° C. to about 35° C. tap water is quickly applied to the product via syringe. Using a circular motion, palms of hands are rubbed together 2 strokes at a time until dissolution occurs (up to 30 strokes). The hand dissolution value is reported as the number of strokes it takes for complete dissolution, 30 strokes as the maximum.

### Process of Forming the Dissolvable Fiber

The process of forming a personal care article may comprise (a) adding one or more water soluble polymers and one or more plasticizers to a twin screw extruder to form a premix; (b) heating the premix to from about 150° C. to about 400° C.; (c) cooling the premix to below 135° C.; (d) mixing one or more anionic surfactants water with the premix to form a mixture; (e) extruding the mixture from the twin screw extruder to produce an extrudate, wherein the extrudate has a moisture content of from about 20% to about 60%, and wherein the extrudate is from about 70° C. to about 130° C.; (f) metering the extrudate through a spinneret assembly to produce one or more fiber strands; (g) spin-drawing and drying the one or more fiber strands to form one or more dissolvable fibers. The one or more dissolvable fibers may have an average diameter of from about 20 microns to about 1,000 microns.

The process of forming a personal care article may comprise adding one or more water soluble polymers and one or more plasticizers to a twin screw to form a premix, and heating the premix to from about 150° C. to about 400° C., alternatively from about 155° C. to about 300° C., and alternatively from about 160° C. to about 250° C. In an embodiment, the one or more water soluble polymers and the one or more plasticizers may be compounded together by a separate extrusion process and then added to the twin screw extrusion process as a single ingredient. In another embodiment, the one or more water soluble polymers and the one or more plasticizers may be added to the twin screw extrusion process as separate ingredients. In an embodiment, a twin-screw extruder from Leistritz (with 27 mm screw diameter, 40:1 L/D ratio, 10 independent temperature control barrel pieces) may be used.

The process of forming a personal care article may comprise cooling the premix to below 135° C., alternatively below about 130° C., alternatively below about 125° C., and alternatively below about 120° C., and then mixing one or more anionic surfactants with the premix to form a mixture. The water may enter the process as a component of one or more raw materials comprising the anionic surfactants, by separate addition to the process, or a combination thereof.

The process of forming a dissolvable fiber may comprise extruding the mixture from the twin screw extruder to produce an extrudate with a defined moisture content range and temperature range. The extrudate may have a moisture content of from about 20% to about 60%, alternatively from about 30% to about 55%, and alternatively from about 40% to about 50%. The temperature range may be from about 70° C. to about 130° C., alternatively from about 80° C. to about 120° C., and alternatively from about 90° C. to about 110° C.

The process of forming a dissolvable fiber may comprise metering the extrudate through a spinneret assembly to produce one or more fiber strands. The spinneret assembly may comprise a distribution plate, a filter block, a meter plate, and a spinneret. The distribution plate may uniformly divert the material flow from the gear pump to the filter block. The filter block may entrap any suspended dirt or particulates, which may pose adverse effects on the spinability of the fibers. The meter plate in between the filter block and the spinneret may further stabilize the flow. The spinneret may comprise an array of nozzles in order to produce multiple fiber strands. In an embodiment, the nozzles may have a nozzle size of from about 0.1 mm to about 3 mm, alternatively from about 0.2 mm to about 2.5 mm, alternatively from about 0.3 mm to about 2.0 mm, and alternatively from about 0.4 mm to about 1.5 mm. The nozzle size is the outer diameter of the nozzle. In an embodiment, the nozzles may also have a trilobal geometery.

The process of forming a dissolvable fiber may comprise spin-drawing and drying the one or more fiber strands within an air circulation column and spun by godget rollers to form one or more dissolvable fibers with an average diameter of from about 20 microns to about 1,000 microns. In an embodiment, the air circulation column may be heated by downstream drying hot air with a temperature of from about 40° C. to about 120° C., alternatively from about 50° C. to about 110° C., and alternatively from about 60° C. to about 100° C. In an embodiment, the one or more godget rollers may be heated to a temperature of from about 60° C. to about 130° C., alternatively from about 70° C. to about 120° C., and alternatively from about 80° C. to about 110° C. In an embodiment, the fibers may be stretched and dried through the air circulation column and godget rollers to form one or more dissolvable fibers with an average diameter of from about 20 microns to about 1,000 microns, alternatively from about 30 microns to about 500 microns, alternatively from about 40 microns to about 250 microns, alternatively from about 50 microns to about 150 microns, and alternatively from about 60 microns to about 100 microns.

In an embodiment, a further zone temperature may be employed involving further cooling of the mixture prior to exiting the extruder or via a secondary tandem extruder. The third zone temperature range may be from about 50° C. to about 110° C., alternatively from about 60° C. to about 100° C., and alternatively from about 70° C. to about 90° C.

In an embodiment, a twin screw extrusion process, either alone or in combination with other forming operations, may be used depending on the desired type of the final product. Two different types of extruders may be employed consisting of a twin screw extruder and single screw extruder. The twin screw extruder may be a conical twin screw extruder. In an embodiment, the process may utilize a tandem extrusion set up which consists of two or more of extruders connected in a series or in parallel. The tandem extrusion set up may use a twin-screw extruder to improve mixing between the water soluble polymer and the rest of ingredients, followed by a single-screw extruder for effective cooling.

### Fiber Spinning

Continuous fibers of different personal care compositions may be synthesized through an extrusion-based fiber spinning process. The process may be initiated with plasticating and homogenizing a mixture in an extruder, optionally a twin-screw intermeshing counter-rotating type extruder. The homogenized flow of the formulation may then be extruded and metered through a spinneret assembly to achieve an array of finer-sized fiber strands of the desired geometry. These fibers may be subsequently spin-drawn and dried into the final fiber dimensions through an array of godet rollers.

Fiber spinning examples may be carried out using a twin-screw extrusion-based fiber spinning system. The system may be comprised of a Brabender twin-screw extruder (with 42 mm diameter, 7:1 L/D ratio, counter-rotating, intermeshing screws) to feed in and plasticate the formulation and a melt spinning gear pump (1.8 cm³ capacity) to deliver a uniform material flow downstream to a spinneret die assembly. The fiber spinning system may be equipped with four temperature monitoring zones: conveying, plasticating, and metering zones in the extruder (T1 to T3), and the gear pump (T_{gear pump}).

At the spinneret assembly, the flow of the plasticated extrudate may be diverted into finer-sized fiber die profiles. In this work, the spinneret assembly may be comprised of a distribution plate, a filter block, a meter plate, and a spinneret. The distribution plate may uniformly divert the material flow from the gear pump to the filter block. The filter block may entrap any suspended dirt or particulates, which may pose adverse effects on the spinability of the fibers. The meter plate in between the filter block and the spinneret may further stabilize the flow and reduce the plastic memory effect experienced by the extrudate. The spinneret may comprise an array of nozzles (trilobal geometry, approximately 1.5 mm in size) to divert the extrudate into multiple fiber strands. An alternative spinneret, which has a smaller nozzle size of 0.5 mm, may also be available for achieving fibers of finer geometries. Fiber strands exiting the spinneret assembly may be naturally stretched by gravity as they travel down the air circulation column and then may be spun by the godet rollers at the base of the fiber spinning system at controlled rates (60 to 2000 m/min).

The air circulation column may be integrated with the capability of delivering hot/ambient air or steam circulation to the fiber strands for maintaining the temperature and water content of the strands. The two godet rollers at the bottom of the system may be equipped with heaters so that the extruded fiber strands may be spun and dried at a desired temperature.

### Preparation of the Extrudate

The extrudate may be prepared through either an inline or a two-pass process. For inline formulation preparation, a water soluble polymer, a plasticizer may be added to the twin-screw extruder of the fiber spinning system at the first zone to form a premix. The surfactant solution, water, and other ingredients will be introduced into a later zone of the extrusion system to homogenize with the premix. The mixture of extrudate will be metered to the spinneret assembly for the subsequent fiber spinning process.

For the two-pass process, the water soluble polymer, plasticizer, surfactant solution, and other ingredients will be compounded into a formulation masterbatch. Water may be added to the masterbatch during the compounding process or in a subsequent step to condition the moisture content of the masterbatch to the desired level. The masterbatch may be added into the extrusion-based fiber spinning system, extruded, and spun into the final fiber geometries.

The extrudate in the fiber spinning examples presented may be prepared through the two-pass process. Unless otherwise specified, the extrudate may be prepared at the indicated weight percentages as described in Table 1 and may be conditioned in a subsequent step with additional water to achieve the desired moisture contents. Pellets of PVOH/glycerin compound may be fed into the extruder using a weight-loss gravimetric feeder at a pre-determined mass flow rate. The aqueous surfactant solution and water may be metered into the twin-screw extruder at Zone 3 and Zone 6, respectively, according to the material composition as described on Table 1. The mixture may then be allowed to cool and set in an ambient environment. The composition of extruded masterbatch prior to the moisture conditioning step is tabulated on Table 2.

TABLE 1
Weight ratios of the ingredient of the masterbatch prior to
moisture conditioning

| Ingredient | Weight Ratio (phr) |
|---|---|
| Polyvinyl alcohol | 100 |
| Glycerin | 50 |
| 70% aq. surfactant solution | 128 |
| Water | 60 |

TABLE 2
Composition of the surfactant masterbatch prior to moisture conditioning

| Ingredient | Composition (% w/w) |
|---|---|
| Polyvinyl alcohol | 29.6 |
| Glycerin | 14.8 |
| Sodium laureth-1 sulfate | 26.6 |
| Water | 29.1 |

Extrudate of different moisture contents may be prepared by conditioning the extruded masterbatch with additional water. Moisture conditioning of the extrudate may be achieved through either of the two following techniques:

(i) saturating the material in a moisturizing chamber; or

(ii) directly sprinkling of water at a pre-determined content onto the material.

Table 3 shows the moisture content of the formulation estimated by measuring the moisture uptake of the masterbatch during the conditioning process. Due to the hygroscopic nature of the processing masterbatch, it is critical to take special considerations about the residual moisture content of the masterbatch in order to determine the amount of additional water that may be introduced to the masterbatch. For examples, the residual moisture content of the masterbatch resided in an environment of different relative humidity (RH) levels and may vary between approximately 11% w/w in a typical dry day (45RH %) and approximately 21% w/w in a typical moist day (63RH %).

TABLE 3
Measured Moisture contents with different conditioning techniques

| | Target Moisture | | Measured Moisture | |
|---|---|---|---|---|
| Technique | Content | | Content | |
| Moisturizing | 50 to 55% | w/w | 47% | w/w |
| Chamber | | | | |
| Direct Sprinkling | 48% | w/w | 46.7% | w/w |
| Direct Sprinkling | 50% | w/w | 50.2 to 52.5% | w/w |

### Fiber Spinning Examples

The following examples further describe and demonstrate embodiments within the scope of the provided invention. The examples are given solely for the purpose of illustration and are not to be construed as limitations, as many variations thereof may be possible. All exemplified amounts are concentrations by weight of the total composition, i.e., wt percentages (% w/w), unless otherwise specified.

### Moisture Conditioning with Moisture Chamber

### Example 1

In this example, four extrudates of different moisture contents were considered. The compounded processing masterbatch was allowed to saturate in a moisturizing chamber to attain the desired moisture contents. Table 4 summarizes the composition of the four extrudates examined in this example. Each of the four extrudates was allowed to be processed with the twin-screw extrusion-based fiber spinning system as described earlier. Table 5 shows the processing conditions that were used in the spinning process. Extruded fiber strands of the extrudates were allowed to be spun with Godet Roller #1 of the system. Table 6 summarizes the characterization results of the spun fibers.

TABLE 4
Composition of the Extrudates

| | Composition (% w/w) | | | |
|---|---|---|---|---|
| Ingredient | Example 4.1 | Example 4.2 | Example 4.3 | Example 4.4 |
| Polyvinyl | 27.1 to 29.2 | 20.8 to | 18.8 to | 15.4 to |
| alcohol | | 22.9 | 20.8 | 18.8 |
| Glycerin | 13.6 to 14.6 | 10.4 to | 9.4 to 10.4 | 7.7 to |
| | | 11.4 | | 9.4 |
| Sodium | 24.3 to 26.2 | 18.7 to | 16.9 to | 13.8 to |
| laureth-1 | | 20.6 | 18.7 | 16.9 |
| sulfate | | | | |
| Water | 30.0 to 35.0 | 45.1 to | 50.1 to | 54.9 to |
| | | 50.1 | 54.9 | 63.1 |

TABLE 5
Processing conditions during the fiber spinning process

| RPMₑₓₜ | RPMₚᵤₘₚ | T1 | T2 | T3 | Tₚᵤₘₚ |
|---|---|---|---|---|---|
| 30 | 15 | 90° C. | 90° C. | 90° C. | 100° C. |

TABLE 6
Characterization results of the spun fibers of each extrudate

| | Example 1.1 | Example 1.2 | Example 1.3 | Example 1.4 |
|---|---|---|---|---|
| Highest | 55 m/min | 95 m/min | 110 m/min | 200 m/min |
| spinning | | | | |
| speed | | | | |
| achieved | | | | |
| Mean spun | 420μ | 400 to 425μ | 300 to 310μ | 95 to 100μ |
| fiber size | | | | |

As the moisture content of the extrudates was increased from approximately 30-35% w/w (Example 1.1) to approximately 55-63% w/w (Example 1.4), the finest achievable size of the spun fibers was reduced from 420μ to 95μ. Extrudates of higher initial moisture contents can be spun into finer fiber geometries under the processing temperature. In order to achieve a spun fiber size of 50-150μ, or 50-100μ at the processing conditions examined, extrudates of higher moisture contents, for example 54.9-63.1% w/w, may be used. It is to be noted that, for higher moisture content extrudates, for instance, Example 1.4, the extruded fiber strands may be relatively humid in nature and may have been thinned considerably by gravity as they travel down the vertical column. Additional downstream drying may become necessary to prevent agglomeration of these fibers.

### Moisture Conditioning with Direct Sprinkling of Water

### Example 2

In this example, the extrudates examined possessed a moisture content of approximately 45, 48, and 50% w/w. Moisture conditioning of the formulations was carried out through directly sprinkling of water at a pre-determined amount onto the surfactant masterbatch. Table 7 summarizes the composition of the extrudate examined in this example. The personal care extrudate in the present example was allowed to be processed with the said twin-screw extrusion-based fiber spinning system as described earlier. Table 8 shows the processing conditions used in the spinning process. Extruded fiber strands of the extrudates were allowed to be spun with Godet Roller #1 of the system at various godet spinning speeds. Table 9 summarizes the characterization results of the spun fibers achieved in this example.

TABLE 7
Composition of the extrudates

| | Composition (% w/w) | | |
|---|---|---|---|
| | Example | Example | Example |
| Ingredient | 2.1 | 2.2 | 2.3 |
| Polyvinyl alcohol | 22.9 | 21.7 | 20.8 |
| Glycerin | 11.5 | 10.8 | 10.4 |
| Sodium laureth-1 | 20.6 | 19.4 | 18.7 |
| sulfate | | | |
| Water | 45.0 | 48.1 | 50.1 |

TABLE 8
Processing conditions during the fiber spinning process

| RPMₑₓₜ | RPMₚᵤₘₚ | T1 | T2 | T3 | Tₚᵤₘₚ |
|---|---|---|---|---|---|
| 10 | 5 | 85° C. | 90° C. | 90° C. | 100° C. |

TABLE 9
Characterization results of the spun fibers of each extrudate

| | Example 4.1 | Example 4.2 | Example 4.3 |
|---|---|---|---|
| Mean spun | >150μ | 125 to 145μ | 75 to 95μ |
| fiber size | | | |

As the moisture content of the extrudates was increased from approximately 45% w/w (Example 2.1) to approximately 50% w/w (Example 2.3), the finest achievable size of the spun fibers was reduced from >150μ to approximately 75-95μ. Similar to Example 1 discussed earlier, the spinability of the extruded fiber strands may depend on the moisture content of the extrudates. The fiber strands attained from extrudate of 50% w/w moisture (Example 2.3) may be relatively humid and occasional discontinuities of the material flow may be experienced.

### Example 3

### Downstream Drying through Hot Air Circulation

In this example, the extrudate examined possessed a moisture content of approximately 48% w/w. Moisture conditioning of the formulations was carried out through directly sprinkling of water at a pre-determined amount onto the surfactant masterbatch. Table 10 summarizes the composition of the extrudate examined in this example. The extrudate in the present example was allowed to be processed with the twin-screw extrusion-based fiber spinning system as described earlier. Table 11 shows the processing conditions that were used in the spinning process. Extruded fiber strands of the extrudates were allowed to be spun with Godet Roller #1 of the system at a godet spinning speed of 160 m/min. In this example, downstream drying was achieved through circulating 70-75° C. hot air along the vertical column in between the spinneret assembly and the Godet Roller #1. Table 12 summarizes the characterization results of the spun fibers that were achieved in this example.

TABLE 10
Composition of the extrudate

| Ingredient | Composition (% w/w) |
|---|---|
| Polyvinyl alcohol | 21.7 |
| Glycerin | 10.8 |
| Sodium laureth-1 sulfate | 19.4 |
| Water | 48.1 |

TABLE 11
Processing conditions during the fiber spinning process

| RPMₑₓₜ | RPMₚᵤₘₚ | T1 | T2 | T3 | Tₚᵤₘₚ |
|---|---|---|---|---|---|
| 10 | 5 | 85° C. | 90° C. | 90° C. | 100° C. |

TABLE 12
Characterization results for the spun fibers attained with hot air circulation

| | Example 3.1 | Example 3.2 |
|---|---|---|
| Air | 70-75° C. | 70-75° C. |
| Temperature | | |
| Circulation | Lower Half | Full Column |
| Mean spun | 110 to 125μ | 170 to 185μ |
| fiber size | | |

It was observed that by incorporating downstream drying, through circulating hot air at the vertical column, relatively dry, tri-lobal spun fibers ranged from 110 to 185μ were attained from the examined 48% w/w-moisture extrudate. In this example, spun fibers of relatively thicker (170 to 185μ) and drier texture may be achieved when the circulation of hot air is introduced to the entire length of the vertical column.

### Example 4

### Downstream Drying through Heated Godet Surface

In this example, the extrudate examined possessed a moisture content of approximately 48% w/w. Moisture conditioning of the formulations was carried out through directly sprinkling of water at a pre-determined amount onto the surfactant masterbatch. Table 13 summarizes the composition of the extrudate examined in this example. The extrudate was allowed to be processed with the twin-screw extrusion-based fiber spinning system as described earlier. Table 14 shows the processing conditions that were used in the spinning process. Extruded fiber strands of the extrudates were allowed to be spun with Godet Roller #1 of the system at various godet spinning speeds. In this example, downstream drying was achieved through heating Godet Roller #1 to an elevated temperature of approximately 52-65° C. Table 15 summarizes the characterization results of the spun fibers that were achieved in this example.

TABLE 13
Composition of the Extrudate

| Ingredient | Composition (% w/w) |
|---|---|
| Polyvinyl alcohol | 21.7 |
| Glycerin | 10.8 |
| Sodium laureth-1 sulfate | 19.4 |
| Water | 48.1 |

TABLE 14
Processing conditions during the fiber spinning process

| RPMₑₓₜ | RPMₚᵤₘₚ | T1 | T2 | T3 | Tₚᵤₘₚ |
|---|---|---|---|---|---|
| 10 | 5 | 85° C. | 90° C. | 90° C. | 100° C. |

TABLE 15
Characterization results for the spun fibers attained with
heated godet roller

| | Example 5 |
|---|---|
| Godet | 200 to 210 m/min |
| spinning | |
| speed | |
| Mean spun | 112 to 128μ |
| fiber size | |

It was observed that by incorporating downstream drying, through a heated godet roller surface, relatively dry tri-lobal spun fibers of 112-128μ were attained from extrudate of 48% w/w moisture.

### Example 5

### Fiber Spinning with Different Godet Spinning Speed

In this example, the extrudate examined possessed a moisture content of approximately 49% w/w. Moisture conditioning of the formulations was carried out through directly sprinkling of water at a pre-determined amount onto the surfactant masterbatch. Table 16 summarizes the composition of the extrudate that may be achieved in this example. The extrudate in the present example was allowed to be processed with the twin-screw extrusion-based fiber spinning system as described earlier. Table 17 shows the processing conditions that were used in the spinning process. Extruded fiber strands of the extrudates were allowed to be spun with Godet Roller #1 of the system at various godet spinning speeds. In this example, downstream drying was achieved through heating Godet Roller #1 to an elevated temperature of 100° C. to promote drying of the spun fibers. Table 18 summarizes the characterization results of the spun fibers that may be achieved with different godet spinning speeds.

TABLE 16
Composition of the extrudate

| Ingredient | Composition (% w/w) |
|---|---|
| Polyvinyl alcohol | 21.3 |
| Glycerin | 10.6 |
| Sodium laureth-1 sulfate | 19.1 |
| Water | 49.0 |

TABLE 17
Processing conditions during the fiber spinning process

| RPMₑₓₜ | RPMₚᵤₘₚ | T1 | T2 | T3 | Tₚᵤₘₚ |
|---|---|---|---|---|---|
| 10 | 5 | 85° C. | 90° C. | 90° C. | 100° C. |

TABLE 18
Characterization results of the spun fibers attained with
different godet speeds

| | Example 5.1 | Example 5.2 | Example 5.3 | Example 5.4 |
|---|---|---|---|---|
| Godet | 215 m/min | 255 m/min | 275 m/min | 285 m/min |
| spinning | | | | |
| speed | | | | |
| Mean spun | 125μ | 121μ | 99μ | 97μ |
| fiber size | | | | |

It was observed that as the spinning speed of Godet Roller #1 is increased from 215 m/min (Example 5.1) to 285 m/min (Example 5.4), the achievable size of the spun fibers was reduced from 125μ to 95μ. It was observable that the drawing-ratio of the fibers of a given extrudate may be controlled by the spinning speed of the godet roller. For the present example with an extrudate of 49% w/w moisture, in order to achieve a spun fiber size of 50-100μ at the processing conditions examined, a godet speed of 275 m/min or higher would be recommended. It is to be noted that the trilobal geometry of the fibers attained from this example may become less pronounced, as compared to that in Example 4. Such minor distortion in fiber geometry may be attributed to the relatively low visco-elasticity the extrudate possesses at the processing conditions examined in this example.

### Example 6

### Fiber Spinning with Smaller Spinneret Nozzles and a Secondary Roller

The spinneret used in this example had a reduced nozzle size (0.5 mm). Extrudate of moisture content approximately 45% w/w was prepared through moisture conditioning with directly sprinkling of water onto the compounded processing masterbatch. Table 19 summarizes the composition of the extrudate examined. The extrudate in the present example was allowed to be processed with the twin-screw extrusion-based fiber spinning system as described earlier. Table 20 shows the processing conditions that were used in the spinning process. To achieve finer fiber geometries, the extruded fiber strands of the extrudates were passed onto the heated surface of Godet roller #1 to dry and fed to a secondary stretching roller or Godet roller #2 for further size reduction and fiber collection. Characterization results of the fibers achieved are summarized on Table 21.

TABLE 19
Composition of the extrudate

| Ingredient | Composition (% w/w) |
|---|---|
| Polyvinyl alcohol | 22.9 |
| Glycerin | 11.5 |
| Sodium laureth-1 sulfate | 20.6 |
| Water | 45.0 |

TABLE 20
Processing conditions during the fiber spinning process

| RPMₑₓₜ | RPMₚᵤₘₚ | T1 | T2 | T3 | Tₚᵤₘₚ |
|---|---|---|---|---|---|
| 20 | 3 | 75° C. | 85° C. | 85° C. | 100° C. |

TABLE 21
Characterization results of the fibers spun with
a secondary stretching roller

| | Example 6.1 |
|---|---|
| Mean spun | 55 to 75μ |
| fiber size | |

A coil of continuous dried fiber of personal care composition of size in the range of 55 to 75μ was achieved with the secondary stretching roller.

### Production of Fibers with Other Surfactant Compositions

### Example 7

### Personal Care Fibers Containing Color Dye and Perfume

The spinneret used in this example had a reduced nozzle size (0.5 mm). Extrudate of moisture content approximately 47% w/w was prepared through moisture conditioning with directly sprinkling of water onto the compounded processing masterbatch. Traces of color dye and perfume were be also added onto the extrudate for the production of scented surfactant fibers of blue, light blue, and green colors. Table 22 shows the composition of the extrudate. The extrudate in the present example was allowed to be processed with the twin-screw extrusion-based fiber spinning system as described earlier. Table 23 shows the processing conditions that may be used in the spinning process. Extruded fiber strands of the extrudates were allowed to be spun with Godet Roller #1 of the system at a speed of 250 m/min. In this example, downstream drying was achieved through heating Godet Roller #1 to an elevated temperature of 60-90° C. to promote drying of the spun fibers for characterization. Table 24 shows the characterization results of the colored and scented fibers achieved.

TABLE 22
Composition of the extrudate

| Ingredient | Composition (% w/w) |
|---|---|
| Polyvinyl alcohol | 21.7 |
| Glycerin | 10.8 |
| Sodium laureth-1 sulfate | 19.5 |
| Water | 46.1 |
| Perfume | 2.0 |
| Color dye | <0.005 |

TABLE 23
Processing conditions during the fiber spinning process

| RPMₑₓₜ | RPMₚᵤₘₚ | T1 | T2 | T3 | Tₚᵤₘₚ |
|---|---|---|---|---|---|
| 10 | 5 | 85° C. | 90° C. | 90° C. | 100° C. |

TABLE 24
Characterization results of the spun colored and scented fibers

| | Example 7.1 | Example 7.2 | Example 7.3 |
|---|---|---|---|
| | (Blue) | (Light Blue) | (Green) |
| Godet | 250 m/min | 250 m/min | 250 m/min |
| spinning | | | |
| speed | | | |
| Mean spun | 145μ | 150μ | 155μ |
| fiber size | | | |

### Example 8

### Personal Care Fibers with Color, Perfume, Betaine, and Silicone Fluid

The processing masterbatch was prepared from compounding the ingredients in Table 25 using a twin-screw compounder. The masterbatch was then moisture conditioned to a moisture content of approximately 47% w/w through the direct water sprinkling technique. The conditioned extrudate in the present example was allowed to be processed with the twin-screw extrusion-based fiber spinning system as described earlier. Spinneret with 0.5 mm-sized nozzles was used in this example. Table 26 shows the processing conditions that were used in the spinning process. Extruded fiber strands of the extrudates were allowed to be spun with Godet Roller #1 of the system at a speed of 255 m/min. In this example, downstream drying was achieved through heating Godet Roller #1 to an elevated temperature of 100° C. to promote drying of the spun fibers. Table 27 summarizes the characterization results of the colored and scented fibers that may be achieved. The dried fibers was evaluated to possess 11.9% moisture under room condition; based on this moisture content, the composition of the spun fibers was estimated and are listed in Table 28.

TABLE 25
Composition of the processing formulation

| Ingredient | Composition (% w/w) |
|---|---|
| Polyvinyl alcohol | 26.9 |
| Glycerin | 13.4 |
| Water | 33.1 |
| Sodium laureth-1 sulfate | 19.2 |
| CAPB (betaine) | 1.4 |
| Silicone fluid | 2.0 |
| Perfume | 4.0 |
| Color dye | <0.005 |

TABLE 26
Processing conditions during the fiber spinning process

| RPMₑₓₜ | RPMₚᵤₘₚ | T1 | T2 | T3 | Tₚᵤₘₚ |
|---|---|---|---|---|---|
| 10 to 15 | 5 to 7 | 85° C. | 90° C. | 90° C. | 100° C. |

TABLE 27
Characterization results of the spun fibers attained

| | Example 8.1 |
|---|---|
| Godet | 255 m/min |
| spinning | |
| speed | |
| Mean spun | 150μ |
| fiber size | |

TABLE 28
Estimated composition of the spun personal care fibers

| Ingredient | Composition (% w/w) |
|---|---|
| Polyvinyl alcohol | 35.4 |
| Glycerin | 17.7 |
| Water | 11.9 |
| Sodium laureth-1 sulfate | 25.3 |
| CAPB (betaine) | 1.9 |
| Silicone fluid | 2.6 |
| Perfume | 5.3 |
| Color dye | <0.006 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited herein are incorporated herein by reference in their entirety; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care article comprising one or more extruded dissolvable fibers, the extruded dissolvable fibers comprising:
a. from about 10% to about 60% of one or more anionic surfactants, by weight of the extruded dissolvable fibers, wherein the one or more anionic surfactants have a Krafft point of less than about 30° C.;
b. from about 10% to about 50% of one or more water soluble polymers, by weight of the extruded dissolvable fibers;
c. from about 1% to about 30% of one or more plasticizers, by weight of the extruded dissolvable fibers; and
d. from about 0.01% to about 30% water, by weight of the extruded dissolvable fibers;
wherein the one or more extruded dissolvable fibers has an average diameter of from about 20 microns to about 1,000 microns; and
wherein the personal care article has a dry density of from about 0.02 g/cm³ to about 0.30 g/cm³.

2. The personal care article of claim 1, wherein the dissolvable fiber has an average diameter of from about 30 microns to about 500 microns.

3. The personal care article of claim 1, wherein the dissolvable fiber has an average diameter of from about 40 microns to about 250 microns.

4. The personal care article of claim 1, wherein the one or more anionic surfactants have a Krafft point of less than about 25° C.

5. The personal care article of claim 1, wherein the one or more anionic surfactants comprises one or more alkyl ether sulfates according to the following structure: [chemical structure]
wherein R¹ is a C-linked monovalent substituent selected from the group consisting of:
a. substituted alkyl systems comprising from about 9 to about 15 carbon atoms;
b. unsubstituted alkyl systems comprising from about 9 to about 15 carbon atoms;
c. straight alkyl systems comprising from about 9 to about 15 carbon atoms;
d. branched alkyl systems comprising from about 9 to about 15 carbon atoms; and
e. unsaturated alkyl systems comprising from about 9 to about 15 carbon atoms;
wherein R² is selected from the group consisting of:
a. C-linked divalent straight alkyl systems comprising from about 2 to about 3 carbon atoms;
b. C-linked divalent branched alkyl systems comprising from about 2 to about 3 carbon atoms; and
c. combinations thereof;
wherein M+ is a monovalent counterion selected from a group consisting of sodium, potassium, ammonium, protonated monoethanolamine, protonated diethanolamine, and protonated triethanolamine; and
wherein x is on average of from about 0.5 moles to about 3 moles.

6. The personal care article of claim 5, wherein x is on average from about 0.5 moles to about 3.0 moles of ethylene oxide.

7. The personal care article of claim 5, wherein the alkyl ether sulfate is sodium laureth sulfate.

8. The personal care article of claim 1, wherein the personal care article comprises two or more layers.

9. The personal care article of claim 1, wherein the personal care article has a hand dissolution value of from about 1 stroke to about 30 strokes.

10. The personal care article of claim 1, wherein the personal care article has a dry density of from about 0.04 g/cm³ to about 0.25 g/cm³.

11. The personal care article of claim 1, wherein the one or more extruded dissolvable fibers comprises from about 15% to about 50% of one or more anionic surfactants.

12. The personal care article of claim 1, wherein the one or more extruded dissolvable fibers comprises from about 1% to about 20% water.

13. The personal care article of claim 1, wherein the one or more water soluble polymers is selected from the group consisting of polyvinyl alcohol, polyvinylpyrrolidone, polyalkylene oxide, starch, starch derivatives, pullulan, gelatin, hydroxypropylmethylcellulose, methycellulose, carboxymethycellulose, and mixtures thereof.

14. The personal care article of claim 1, wherein the one or more plasticizers is selected from the group consisting of glycerin, propylene glycol, polyols, copolyols, polycarboxylic acids, polyesters, dimethicone copolyols, and mixtures thereof.

15. The personal care article of claim 1, wherein the one or more extruded dissolvable fibers further comprises a secondary surfactant selected from the group consisting of amphoteric surfactants, zwitterionic surfactants, and mixtures thereof; and wherein the ratio of the one or more anionic surfactants to the secondary surfactant is from about 10:1 to about 1:2.

16. The personal care article of claim 1, wherein the one or more extruded dissolvable fibers further comprises from about 0.1% to about 15% of one or more benefit agents.

17. The personal care article of claim 16, wherein the one or more benefit agents are selected from the group consisting of anti-dandruff agents, conditioning agents, moisturizers, and combinations thereof.

18. The personal care article of claim 17, wherein the conditioning agent is selected from the group consisting of silicones, aminosilicones, quaternized silicones, and combinations thereof.

19. The personal care article of claim 1, wherein the dissolvable fiber further comprises a cationic polymer.

20. The personal care article of claim 1, wherein the one or more extruded dissolvable fibers is trilobal.
